(19) United States
(12) Patent Application Publication
(10) Pub. No.: US 2025/0326701 A1
(43) Pub. Date: Oct. 23, 2025

(54) CONVERSION OF CARBON OXIDES TO SUSTAINABLE GASOLINE

(71) Applicant: TOPSOE A/S, Kgs. Lyngby (DK)

(72) Inventors: Mathias JØRGENSEN, Kgs. Lyngby (DK); Rickard VANNBY, Kgs. Lyngby (DK)

(73) Assignee: TOPSOE A/S, Kgs. Lyngby (DK)

(21) Appl. No.: 18/871,001

(22) PCT Filed: Jun. 15, 2023
(86) PCT No.: PCT/EP2023/066094

§ 371 Date: Dec. 2, 2024

(30) Foreign Application Priority Data
Jun. 20, 2022 (EP) 22179907.5

(51) Int. Cl. C07C 1/12; B01J 6/00; B01J 8/02; B01J 19/24; C10L 1/04; C25B 15/08

(52) U.S. Cl. CPC C07C 1/12; B01J 6/00; B01J 8/0278; B01J 19/245; C10L 1/04; C25B 15/081; C10L 2200/0423

(57) ABSTRACT

A reforming system is provided for autothermal reforming of a by-product stream rich in paraffins of a gasoline synthesis plant incorporating the reforming system. The invention provides an overall more efficient feed-to-gasoline system and process.

### TECHNICAL FIELD

The present invention relates to a more efficient system (plant) and process for producing gasoline from a carbon oxide containing feed, such as a carbon dioxide rich feed. The plant or process comprises methanol synthesis, gasoline synthesis and optional upgrading of the gasoline. Embodiments of the invention include the provision of stand-alone autothermal reforming (ATR) to improve gasoline yield by feeding the ATR with a by-product stream rich in paraffins, such as a propane and/or butane rich stream, and/or off-gas streams, which are produced in the gasoline synthesis plant.

### BACKGROUND

Processes for the conversion of sustainable feeds, such as CO₂, biomass etc. to gasoline or jet fuel via methanol are known. Biomass can first be converted to syngas via gasification followed by conversion of said syngas to methanol, for instance in a methanol loop, and finally methanol conversion to gasoline. CO₂ feed, together with H₂ feed, can be converted to methanol followed by conversion of said methanol to gasoline. Irrespective of the main feed, there are some by-products along with gasoline. One of the by-products from such processes is a fraction rich in paraffins, for instance a stream rich in propane and/or butane (C3 and/or C4). A propane and/or butane stream is known as liquified petroleum gas, LPG. Off-gas streams comprising CO₂, H₂, CH₄, higher hydrocarbons etc. are also typically produced.

The stream rich in paraffins may often itself be considered to have little commercial value. Moreover, the off-gas streams often have no efficient use, apart from using them in fired equipment, which causes CO₂ emission. It would, therefore, be of interest to recycle these product streams as part of the gasoline synthesis process itself, in order to at least improve overall carbon efficiency (C-efficiency) of this process. It would also be desirable to be able to enhance methanol synthesis, e.g. methanol loop, performance of the gasoline synthesis plant and thereby also the yield of gasoline produced.

A stream rich in paraffins, including a stream rich in propane and/or butane such as an LPG stream, and/or off-gas streams can be subjected to a traditional reforming process, such as steam methane reforming, and the reformed synthesis gas stream can be recycled to the methanol loop. In gasoline synthesis from methane, the plant should already comprise a reformer and, thus, LPG and/or off-gas streams could be directed there. However, plants/systems for gasoline synthesis from sustainable feeds and/or feeds from biogas gasification and/or mixtures comprising CO₂ and H₂ do not comprise a reformer.

EP 3730473 A1 discloses the use of renewable energy in methanol synthesis plant. Steam reforming of a number of hydrocarbon feedstocks including LPG is provided in a syngas generation section upstream methanol synthesis to provide for the methanol synthesis gas, and which is further configured such that more of the net energy required by i.a. the methanol synthesis plant, is provided by a non-carbon based energy source, a renewable energy source, and/or electricity.

WO 2010143980 discloses a system for integrating methanol production and hydroprocessing of oil feedstock to produce a hydrocarbon product. A steam reformer processes a first feedstock and C1 to C4 hydrocarbons may be separated from the hydrocarbon product and recycled to the first feedstock. Hence, the steam reformer is provided upstream methanol synthesis to provide for the main methanol synthesis gas feed, and the hydroprocessing plant, having its own feed oil feedstock, is integrated by benefiting from the hydrogen produced in the methanol synthesis.

Applicant's US2020109051 discloses a process for preparing synthesis gas combining electrolysis of water, tubular steam reforming i.e. steam methane reforming (SMR), and autothermal reforming of a hydrocarbon feed stock. The synthesis gas may in a further step be converted to a methanol product.

Applicant's US2022041440 discloses a process for preparing synthesis gas combining electrolysis of carbon dioxide, optional tubular steam reforming i.e. steam methane reforming (SMR), and autothermal reforming of a hydrocarbon feed stock. The synthesis gas may in a further step be converted to a methanol product.

Applicant's co-pending European patent application No. 22166260.4 discloses the conversion of carbon dioxide to gasoline using electrical steam methane reforming (e-SMR) of a recycled liquified petroleum gas (LPG) stream.

US201213452073A discloses a process and system for producing high octane fuel from carbon dioxide and water. A reforming unit downstream of and in fluid communication with the gasoline or diesel generation unit is arranged to e.g. steam-reform a recycle stream having a significant portion of LPG and fuel gas, namely 15-40 wt % of the liquid product.

WO 2016094138 discloses a single-loop synfuel generation for production of gasoline. FIG. 4 therein discloses a methanol-to-gasoline plant in which a by-product stream is withdrawn and converted to a synthesis gas in a reformer. This synthesis gas is combined with a main synthesis gas and fed to a first reactor for conversion to methanol. LPG and similar off-gases are directed away from the reformer.

In a conventional methanol-to-gasoline plant the loss of carbon in purge streams, off-gases and by-products is approximately 20% of the carbon fed to the process. This loss of carbon is directly associated with loss of hydrogen. A hydrogen balance shows that approximately 20% of the hydrogen contained in hydrocarbons formed in the process is lost in by-products and off-gases withdrawn in the process. One major source of carbon and hydrogen loss is in the C3 and C4 fraction, which cannot be blended into the final gasoline product due to vapor pressure requirements. The C3 and C4 hydrocarbons are taken out as an “LPG” stream which does not fulfil typical LPG specifications and therefore it often has little or no commercial value. Further, waste gas streams generated in the plant or process, hereinafter also referred to as “off-gas streams” or simply “off-gases”, are typically combusted in a furnace to generate heat in the process. The combustion of the off-gases causes emission of carbon dioxide and release heat in excess of what is needed for the process.

More generally, sustainable production of hydrocarbon fuels from carbon neutral sources is foreseen to play an important role in the energy transition going from fossil-based fuels to carbon-neutral fuels. The so-called Power-to-X route for sustainable fuel production is a technically feasible process for obtaining hydrocarbon fuels from hydrogen produced by water and/or steam electrolysis and CO₂ collected from industrial sources or captured from atmospheric air. One particular process is the production of synthetic gasoline going via methanol synthesis from H₂ and CO₂ followed by methanol-to-gasoline (MTG) synthesis. Such process enables production of gasoline from renewable electricity. The main power requirement in such process is the power consumed by an electrolyzer producing the hydrogen feed to the process. Efficient conversion of hydrogen to desired gasoline product is therefore key to the economic viability of such process. Inevitably, there will be loss of hydrogen through by-products and off-gases in the process. By-products such as LPG may be of very little value, as mentioned above, due to limited off-take and/or excessive upgrading required to achieve adequate by-product quality. Therefore, by-products and off-gases are considered as a loss in the process. This loss is proportional to loss of valuable electrolysis hydrogen from the process. Recovery of lost hydrogen would greatly improve the economics of such process.

### SUMMARY

The present invention enables recovery of lost hydrogen as well as carbon in by-products and/or off-gases by conversion of these streams into synthesis gas via a dedicated autothermal reformer. This process enables significantly higher product-to-hydrogen efficiency in a Power-to-X process, here a power-to-gasoline process; or where the synthesis gas is produced from a biomass feedstock.

It has been determined that a stream rich in paraffins, such as LPG, and/or off-gas stream which is recycled, can be enabled to provide higher efficiency of sustainable feed to gasoline conversion. With the proposed plant layout, this can be achieved with significantly lower CO₂ emission compared to traditional processes for a similar purpose. Furthermore, the proposed layout also has provided a possibility of reducing the consumption of hydrogen feedstock, thereby increasing hydrogen-efficiency. Moreover, reformed syngas to methanol synthesis e.g. in a methanol synthesis loop (MeOH loop) ensures a molar ratio of CO/CO₂ that results in lower catalyst volume in the methanol synthesis reactor and thereby, a smaller MeOH loop, as it will become apparent from the description below.

A gasoline synthesis plant is therefore provided, which comprises:
a first CO₂ rich feed comprising CO₂ to said plant, a first H₂ rich feed comprising H₂ to said plant; or a first syngas feed which combines the first CO₂ rich feed and the first H₂ rich feed; or a second syngas feed comprising a carbon oxide and hydrogen to said plant;
a methanol synthesis unit;
a gasoline synthesis section;
optionally, an upgrading section, which comprises a hydroisomerisation (HDI) reactor, optionally a hydrocracking (HCR) reactor;
the gasoline synthesis plant further comprises a reforming system for reforming a by-product stream rich in paraffins from the gasoline synthesis section and/or from the upgrading section, to provide an ATR-based syngas stream; and in which the reforming is autothermal reforming in a stand-alone autothermal reformer (ATR);
and the gasoline synthesis plant is further arranged to feed at least a portion of said ATR-based syngas stream to the inlet of the methanol synthesis unit.

Also provided is a process for gasoline synthesis from a sustainable feed, in such a plant.

Further details of the technology are provided in the enclosed dependent claims and figures.

### LEGENDS TO THE FIGURES

The technology is illustrated by means of the following schematic illustrations, in which:

FIG. 1 shows an embodiment of the reforming system of the invention.

FIG. 2 shows an embodiment of a gasoline synthesis plant, comprising an embodiment of the reforming system of the invention.

FIG. 3 shows another embodiment a gasoline synthesis plant, comprising the reforming system of the invention.

FIG. 4 shows an embodiment of the gasoline synthesis plant, further comprising the provision of excess hydrogen from the methanol synthesis unit being fed to the reforming system.

### DETAILED DISCLOSURE

Unless otherwise specified, any given percentages for gas content are % by volume. All feeds are preheated as required.

The term “synthesis gas” (abbreviated to “syngas”) is meant to denote a gas comprising hydrogen and a carbon oxide, and optionally small amounts of other gasses, such as argon, nitrogen, methane, etc.

The term “a carbon oxide” means CO and/or CO₂.

The term “first syngas feed” means a syngas rich in H₂ and CO₂ resulting from the combination of the first H₂ rich stream and the first CO₂ stream. For instance, the first syngas feed contains about 75 H₂ % and about 25% CO₂ with less than 1% CO.

The term “second syngas feed” means a separate syngas feed produced upstream the methanol synthesis unit of the gasoline synthesis plant. For instance, the second syngas feed comprises H₂ and the carbon oxide(s) in a molar ratio of at least 3:1.

A “sustainable feed” may be a CO₂ feed, a H₂ feed, or combination thereof; or a biomass feed, or a syngas feed produced at least partly from electrolysis.

The term “stand-alone ATR” means an ATR alone or together with an upstream pre-reformer. There is no primary reformer arranged together with the ATR, such as a steam methane reformer (SMR) e.g. a conventional tubular reformer, arranged upstream the ATR.

The term “first, second or third or fourth ATR-based syngas stream”, or more generally “ATR-based syngas stream” means a syngas stream withdrawn from the reforming system, and which comprises the ATR. The ATR-based syngas stream is rich in H₂, CO and CO₂. For instance, the ATR-based syngas stream contains about 60% H₂, 30% CO and 8% CO₂, with the balance being CH₄ and inerts.

The term “reforming” and “steam reforming” are used interchangeably.

The term “at least a portion” of a given stream, means the entire stream or a portion thereof.

The terms “system”, “plant” i.e. process plant, are used interchangeably. Throughout this specification, the term system is used for the reforming, hence the term “reforming system”.

The terms “section” and “unit” refers normally in this specification to a subset of a plant or system.

The use of the article “a” or “an” in connection with an item such as a unit means “one or more”.

The term “suitably” may be given the same meaning as “optionally”, i.e. an optional embodiment.

Other definitions are provided throughout the patent application in connection with the recital of embodiments.

In a first embodiment, a gasoline synthesis plant is provided, which comprises:
a first CO₂ rich feed (201) comprising CO₂ to said plant, a first H₂ rich feed 202 comprising H₂ to said plant, or a first syngas feed 209 combining the first CO₂ rich feed 201 and the first H₂ rich feed 202; or a second syngas feed 205 comprising a carbon oxide and hydrogen to said plant;
a methanol synthesis unit 220, arranged to receive the first CO₂ rich feed 201 and the first H₂ rich feed 202, or arranged to receive the first syngas feed 209, or arranged to receive the second syngas feed 205, and provide an effluent stream 221 comprising methanol;
a gasoline synthesis section 230, arranged to receive at least a portion of the effluent stream 221 comprising methanol, and provide a raw product 231 containing hydrocarbons boiling in the gasoline range;
optionally, an upgrading section 240, arranged to receive at least a portion of the raw product 231 from the gasoline synthesis section 230, and provide a gasoline product stream 241; said optional upgrading section 240 comprising: a hydroisomerisation (HDI) reactor, optionally a hydrocracking (HCR) reactor, thereby providing said gasoline product stream 241;
said gasoline synthesis section 230 and/or said upgrading section 240 being arranged to provide a by-product stream 242, 242′ rich in paraffins;
said gasoline synthesis plant 200 further comprising a reforming system 100 for reforming said by-product stream 242, 242′ rich in paraffins, said reforming system 100 comprising: a first reforming feed stream 1 as said by-product stream 242, 242′ rich in paraffins; an autothermal reformer (ATR, 40) with no primary reformer arranged together with the ATR 40, the ATR 40 being arranged to receive the by-product stream 1, 242, 242′ rich in paraffins and carry out an autothermal reforming step; and said reforming system 100 providing an ATR-based syngas stream 41, 51, 53, 62;
said gasoline synthesis plant (200) further being arranged to feed at least a portion of said ATR-based syngas stream 41, 51, 53, 62 to the inlet of the methanol synthesis unit 220;
said gasoline synthesis plant 200 does not comprise a reforming unit arranged upstream the methanol synthesis unit 220 for providing said first 209 or second 205 syngas feed.

In an embodiment, the ATR-based stream is a first, second or third ATR-based syngas stream 41, 51, 53.

In the reforming system of the gasoline plant, there is no primary reformer arranged together with the ATR, such as a steam methane reformer (SMR) i.e. tubular reformer arranged upstream the ATR, or a convection heated reformer such as a heat exchange reformer arranged in series or in parallel with the ATR. Yet, a pre-reformer (pre-reforming unit) is suitably arranged upstream the ATR. Such an arrangement is also referred to stand-alone ATR, i.e. ATR alone or together with an upstream pre-reformer.

A much simpler plant with significantly lower carbon footprint is thereby provided, as the reforming is only conducted in a minor syngas stream, namely the by-product stream rich in paraffins, optionally also an off-gas stream. Furthermore, the reformed-based syngas to methanol synthesis unit (e.g. MeOH loop, herein also referred to as methanol synthesis loop) ensures a CO/CO₂ molar ratio which is needed for lower catalyst volume and thereby, smaller methanol synthesis unit, e.g. smaller MeOH loop.

The gasoline synthesis plant therefore comprises, in general terms:
a first CO₂ rich feed to said plant, a first H₂ rich feed to said plant, or a first syngas feed combining these streams, or
a second syngas feed comprising a carbon oxide and hydrogen to said plant;
a methanol synthesis unit with no upstream steam reforming unit to provide the second syngas feed;
a gasoline synthesis section;
optionally, an upgrading section; and
a reforming system comprising a stand-alone ATR.

The invention enables increased hydrogen-to-gasoline efficiency as well as increased carbon efficiency while generating heat for the process, and furthermore, there is utilization of the oxygen that is formed as a by-product in the electrolyser, thereby turning this oxygen into a valuable stream from the electrolyser. The need of providing a costly and large unit for producing oxygen, typically an air separation unit (ASU) is eliminated.

On an overall basis, the production rate of methanol and gasoline product increases by 15-25% with the same amount of hydrogen feed flow rate to the process. At the same time, the emission of carbon dioxide from combustion in furnace(s) for process heating is minimized. Addition of ATR-based synthesis gas leads to increased reactivity of the synthesis gas entering the methanol reactor of the methanol synthesis unit due to high carbon monoxide content as compared to the hydrogen and carbon dioxide mixture resulting from combining the first H₂ rich feed and first CO₂ rich feed. The increased reactivity of the synthesis gas gives several advantages: a) even though the methanol production rate increases by 15-25%, e.g. 20% and a higher catalyst volume would thereby be expected, the catalyst volume can be kept the same as what would be needed to convert solely the mixture of hydrogen and carbon dioxide to methanol; b) the water formation in the methanol synthesis is essentially the same as for a base case where solely the mixture of the first H₂ rich feed and first CO₂ rich feed is being fed to the methanol synthesis unit; hence, the boost in production by recycling a by-product stream rich in paraffins and off-gases can be achieved without increasing size and duty considerably in the distillation section used for removing water from the raw methanol being produced in the methanol synthesis unit; c) the maximum stoichiometrically achievable hydrogen efficiency of the methanol synthesis unit, e.g. methanol synthesis loop, increases from about 65% with solely hydrogen and carbon dioxide feed to 75-80% due to reduced loss of hydrogen in water formation in the loop.

The gasoline synthesis plant does not comprise a reforming unit arranged upstream the methanol synthesis unit for providing said first or second syngas feed. Optionally, however, the gasoline synthesis plant may be provided with a reverse water gas shift unit (rWGS unit) arranged upstream the methanol synthesis unit for preparing said second syngas feed, as it also will become apparent from a below embodiment.

Traditionally, steam reforming of a major hydrocarbon feed stream, such as natural gas, is required upstream the methanol synthesis unit for providing a methanol synthesis gas as said first or second syngas feed. The steam reforming unit is typically highly costly in terms of capital and operating expenses, and not least also with a significant carbon footprint. The present application obviates this and instead integrates a reforming system including autothermal reforming which is only dedicated to process a minor hydrocarbon stream into an ATR-based syngas, namely the first stream rich in paraffins from the gasoline synthesis section or upgrading section of the plant, optionally one or more off-gas streams. The sum of these streams still represents a minor stream being fed to the reforming system. For instance, up to 50% (vol. basis), such as 5-45%, is from the reformer-based syngas stream of the reforming system of the plant, as it will also become apparent from a below embodiment. Apart from the benefits associated with enabling a smaller methanol loop, a much smaller reformer is thus required, thereby reducing plant plot size and associated capital and operating expenses.

In an embodiment,
the methanol synthesis unit 220 is arranged to provide an excess hydrogen stream 255, i.e. excess hydrogen stream from the methanol synthesis unit 220, and said optional upgrading section 240 is arranged to receive: a portion of the first or second H₂ rich feed 202, 202′ comprising H₂, and/or said excess hydrogen stream from the methanol synthesis unit 220, such as a portion of said excess hydrogen stream 255 from the methanol synthesis unit 220;
   and/or
the methanol synthesis unit 220 is arranged to provide an excess hydrogen stream 255, i.e. excess hydrogen stream from the methanol synthesis unit 220, and said reforming system 100 is arranged to receive said excess hydrogen stream from the methanol synthesis unit 220, such as a portion of said excess hydrogen stream 255 from the methanol synthesis unit 220; preferably, wherein said reforming system 100 comprises a hydrogenation section 10, and wherein said hydrogenation section 10 is arranged to receive said excess hydrogen stream 255.

Hence, the methanol synthesis unit is suitably arranged to provide an excess hydrogen stream. Thereby, hydrogen required for e.g. the HDI reactor of the upgrading section or for the hydrogenation section in the reforming system of the plant, is sourced internally, rather than costly external sourcing from outside the battery limits of the plant. The excess hydrogen stream from the methanol synthesis unit is suitably generated from the overhead recycle stream from the methanol synthesis unit this suitably also being provided as a methanol synthesis lop (MeOH loop), for instance by providing a hydrogen recovery unit, such as a pressure swing adsorption (PSA) unit arranged to receive a portion of the overhead recycle stream, as described farther below in the present application.

In an embodiment, the gasoline synthesis section 230 comprises: a methanol-to-gasoline section (MTG section) and said downstream upgrading section (240). The upgrading section comprises a distillation section comprising a de-ethanizer and a LPG-splitter; optionally a HDI and/or HCR reactor.

In an embodiment, the upgrading section 240 is arranged to receive: a portion of the first or second H₂ rich feed 202, 202′ comprising H₂, and/or an excess hydrogen stream from the methanol synthesis unit 220, such as a portion of said excess hydrogen stream from the methanol synthesis unit 220.

More specifically, in an embodiment,
optionally, the gasoline synthesis section 230 comprises: a methanol-to-gasoline section (MTG section), and a downstream distillation section comprising a de-ethanizer and a LPG-splitter;
said HDI reactor and/or optional HCR reactor of said optional upgrading section 240 is arranged to receive: a portion of the first or second H₂ rich feed 202, 202′ comprising H₂, and/or an excess hydrogen stream from the methanol synthesis unit 220, such as a portion of said excess hydrogen stream from the methanol synthesis unit 220.

In an embodiment, said reforming system 100 is further arranged for said first reforming feed 1, 242, 242′, i.e. as said by-product stream 242, 242′ rich in paraffins, being less than 15 wt % of said of said raw product 231 containing hydrocarbons boiling in the gasoline range, or less than 15 wt % of said gasoline product stream 241.

The by-product formation and off-gas formation of light hydrocarbons streams in the gasoline synthesis plant represent less than 15 wt %, such as 10 wt % or less, for instance 5 wt % of the gasoline being produced, this being the raw product containing hydrocarbons boiling in the gasoline range, or the gasoline product stream. Despite the by-product and off-gas(es) only representing less than 15 wt % or less, e.g. about 10 wt %, or 5 wt %, of the hydrocarbon product, it is reused in the plant or process to increase its overall efficiency: carbon (C-efficiency) and hydrogen efficiency (H-efficiency). Despite the low percentage of e.g. by-product, a dedicated reforming unit for reforming such by-product and off-gas into a syngas, is advantageously provided.

The first CO₂ rich feed is provided to the methanol synthesis unit (as mentioned before, this suitably being a methanol synthesis loop, or simply methanol loop i.e. MeOH loop). In a particular embodiment, the first CO₂ rich feed comprises more than 75% CO₂, such as more than 90% CO₂, for instance more than 95% CO₂, such as more than 99% CO₂. The first CO₂ rich feed may in addition to CO₂ comprise minor amounts of, for example, steam, oxygen, nitrogen, oxygenates, amines, ammonia, carbon monoxide, and/or hydrocarbons. The first CO₂ rich feed suitably comprises only low amounts of hydrocarbon, such as for example less than 5% hydrocarbons or less than 3% hydrocarbons or less than 1% hydrocarbons.

The first H₂ rich feed is provided to the methanol synthesis unit. Suitably, the first H₂ rich feed consists essentially of hydrogen. The first H₂ rich feed of hydrogen is thus suitably “hydrogen rich” meaning that the major portion of this feed is hydrogen; i.e. over 75%, such as over 85%, preferably over 90%, more preferably over 95%, even more preferably over 99% of this feed is hydrogen.

One source of the first H₂ rich feed of hydrogen may be one or more electrolyser units.

Accordingly, in an embodiment, the gasoline synthesis plant further comprises:
an electrolysis section comprising:
   an electrolysis unit 260 arranged to receive a water feedstock 203, i.e. steam and/or water, and provide: a second H₂ rich feed 202′ comprising H₂; optionally as said first H₂ rich feed 202 comprising H₂, or a portion thereof as said first H₂ rich feed 202; and a first oxygen stream 206;
   and/or
   an electrolysis unit 250 arranged to receive a second CO₂ rich feed 201′, or said first CO₂ rich feed 201 comprising CO₂, or a portion thereof, and provide a CO-enriched feed 204 and a second oxygen stream 206′;
means such as a mixing unit or junction to combine the first or second H₂ rich feed 202, 202′ comprising H₂, with the CO-enriched feed 204, and provide said second syngas feed 205.

In a particular embodiment, the gasoline synthesis plant further comprises:
means such as a mixing unit or junction to combine a steam stream 207 with the first oxygen stream 206 to provide an oxidant stream 208; and wherein the ATR 40 of the reforming system 100 is arranged to receive said oxidant stream 208;
optionally, means such as mixing unit or junction to combine the first 206 and/or second 206′ oxygen streams, with said steam stream 207 to provide oxidant stream 208.

Thereby there is provided a high integration of process streams in the gasoline synthesis plant, while at the same time eliminating the need of providing a large and expensive air separation unit (ASU), typically required for the generation of the oxygen needed in an ATR.

Suitably also, a portion of the second CO₂ rich feed 201′, or a portion of the first CO₂ rich feed 201, may bypass the electrolysis unit 250 and combine with the CO-enriched feed stream 204. Some CO₂ is required in the syngas feed, here the second syngas feed, so the by-pass enables adjusting the syngas feed module and CO₂ content for optimum performance of the methanol synthesis unit. In a particular embodiment, up to 10% of the first or second CO₂-rich feed bypasses the electrolysis unit.

In addition to hydrogen, the first or second H₂-rich feed may for example comprise steam, nitrogen, argon, carbon monoxide, carbon dioxide, and/or hydrocarbons. In some cases, a minor content of oxygen may be present in this first or second H₂ rich feed, typically less than 100 ppm. The first or second H₂ rich feed suitably comprises only low amounts of hydrocarbon, such as for example less than 5% hydrocarbons or less than 3% hydrocarbons or less than 1% hydrocarbons.

Suitably, there is also provided a purification section to remove impurities such as oxygen and hydrocarbons from the first or second H₂ rich feed. Suitably also, there is a purification section to remove impurities such as sulfur containing compounds e.g. COS, from e.g. the first CO₂ rich feed.

The first CO₂ rich feed and the first H₂ rich feed are—in one aspect—combined into said first syngas feed prior to being fed to the methanol synthesis unit.

In this embodiment, the gasoline synthesis plant comprises a methanol synthesis unit being arranged to receive the first CO₂ rich feed and first H₂ rich feed, or the first syngas feed which combines the first CO₂ rich feed and first H₂ rich feed, as well as the ATR-based syngas from the reforming system, suitably the second ATR-based syngas therefrom. An effluent stream comprising methanol is obtained. The process of converting the first CO₂ rich and first H₂ rich streams can occur, for example by compressing them in a first syngas feed compressor and sending the compressed, combined gas as first syngas feed through e.g. a boiling water methanol reactor as one embodiment of a methanol reactor in the methanol synthesis unit, and where at least a portion of the CO, CO₂ and H₂ is converted to methanol followed by a condensation section separating the purge gas stream from the methanol in a liquid phase.

The raw methanol stream, i.e., the effluent stream comprising methanol, comprises a major portion of methanol; i.e. over 50 wt %, such as over 75 wt %, preferably over 85 wt %, more preferably over 90 wt % of this feed is methanol. Other minor components of this stream include but not limited to, higher alcohols, ketones, aldehydes, DME, organic acids and dissolved gases. The raw methanol comprises also water, which normally requires removal e.g. by distillation in order to purify the stream into a stream comprising more than 90% wt methanol. Hence, a water separation section is suitably located between the methanol synthesis unit (220) and the gasoline synthesis section (230), and being arranged to remove water from the effluent stream (221) comprising methanol.

A second syngas feed comprising a carbon oxide and hydrogen may also be provided to the methanol synthesis unit. This second syngas feed is suitably provided by combining the second H₂ rich feed and the CO-enriched feed generated in the electrolysis section.

This also ensures a higher molar ratio CO/CO₂ to the methanol synthesis unit which is superior than providing a CO₂-rich feed, by enabling a lower catalyst volume, less water formation and thus less need for purification downstream to remove it, and thereby also a smaller methanol synthesis unit e.g. MeOH loop. More specifically, the provision of much higher content of CO with respect of CO₂ in the syngas feed, with the molar ratio CO/CO₂ being greater than 1, such as greater than 2, for instance a ratio of 10 or higher, means a more reactive syngas, since it enables that the methanol reaction proceeds with low generation of water which is detrimental for the methanol synthesis catalyst in the subsequent methanol synthesis unit, as the methanol synthesis is conducted mainly according to the reaction: CO+2 H₂═CH₃OH, rather than typically via the reaction 3 H₂+CO₂═CH₃OH+H₂O. Less hydrogen consumption, by a drastic decrease now requiring 2 moles of H₂ instead of 3 moles of H₂ for each mole of produced methanol, is also achieved. The resulting water has also a negative effect on the performance of the methanol synthesis catalyst and the catalyst volume may increase with more than 100% if the CO₂ concentration in the syngas feed is too high, e.g. 90%. Much more energy is also required for the downstream purification of the raw methanol because all the water is removed by distillation. Not least, there is an increase in the output of methanol being produced, which manifests itself in a higher yield of the desired gasoline product.

In an embodiment, the gasoline synthesis plant further comprises:
a thermal decomposition unit, such as a gasification unit, arranged to receive a biomass feedstock to provide a crude syngas feed, and a crude syngas purification section arranged to receive the crude syngas feed and to provide said second syngas feed 205.

The thermal decomposition unit is not a primary reforming unit. It would be understood that the latter, e.g. SMR (tubular reformer), or a convection heated reformer such as a heat exchange reformer, comprises a catalyst arranged as a fixed bed for converting a hydrocarbon feed gas into syngas.

Typically, where there is a gasification for producing a syngas feed for downstream methanol production, the syngas feed is subjected to a shifting step i.e. water gas shift step (WGS step) in a WGS section for changing the composition of the syngas according to the reaction CO+H₂O═CO₂+H₂. The WGS may either be sweet (without sulfur in the syngas) our sour (including sulfur in the syngas). Finally, some of the CO₂ is removed in a CO₂ removal section, which is a highly large unit with concomitant high capital and operating expenses. Further, this results in venting CO₂ from the process. For adjusting of the module M=(H₂−CO₂)/(CO+CO₂) of the syngas feed to the desired level of about 2.0 for the downstream methanol synthesis, typically part of the syngas bypasses the WGS step and the CO₂ removal.

By the present invention, the second syngas feed may be combined with e.g. the first, second or third ATR-based syngas stream of the reforming system, to adjust the feed to the methanol synthesis unit. The need of a WGS section and CO₂-removal section on the second syngas feed to the methanol synthesis unit may thus be eliminated.

The term “thermal decomposition” means any decomposition process, in which a material is partially decomposed at elevated temperature, typically 250° C. to 800° C. or perhaps 1000° C., in the presence of sub-stoichiometric amount of oxygen (including no oxygen). The product will typically be a combined liquid and gaseous stream, as well as an amount of solid char. The term shall be construed to included processes known as gasification, pyrolysis, partial combustion, or hydrothermal liquefaction.

In a particular embodiment, the thermal decomposition is gasification. Thus, the thermal decomposition unit is a gasification unit. The gasification is suitably conducted under the presence of a gasification agent such as oxygen, steam, carbon dioxide, or a combination thereof. Suitably also, the gasification agent is produced in the process; for instance, oxygen is provided by electrolysis and steam from the methanol conversion step in the methanol synthesis unit.

In the crude syngas purification section, under the addition of e.g. water, impurities such as heavy metals, silica, sulfur, which may be detrimental for downstream units and corresponding process steps are removed.

The term “biomass feedstock” means renewable feed, in particular a solid renewable feed. The solid renewable feed is:
a lignocellulosic biomass including: wood products, forestry waste, and agricultural residue; and/or
refused derived fuel (RDF) including municipal waste, i.e. municipal solid waste, in particular the organic portion thereof.

The term “lignocellulosic biomass” means a biomass containing, cellulose, hemicellulose and optionally also lignin. The lignin or a significant portion thereof may have been removed, for instance by a prior bleaching step. The lignocellulosic biomass is forestry waste and/or agricultural residue and comprises biomass originating from plants including grass such as nature grass (grass originating from natural landscape), wheat e.g. wheat straw, oats, rye, reed grass, bamboo, sugar cane or sugar cane derivatives such as bagasse, maize and other cereals.

The term “refused derived fuel (RDF)” means a fuel produced from various types of waste, such as municipal solid waste (MSW), industrial waste or commercial waste. In accordance with the definition provided by Wikipedia.org as of 25 Apr. 2022, RDF consists largely of combustible components of such waste, as non-recyclable plastics (not including PVC), paper cardboard, labels, and other corrugated materials. These fractions are separated by different processing steps, such as screening, air classification, ballistic separation, separation of ferrous and non-ferrous materials, glass, stones and other foreign materials and shredding into a uniform grain size, or also pelletized in order to produce a homogeneous material which can be used as substitute for fossil fuels in e.g. cement plants, lime plants, coal fired power plants or as reduction agent in steel furnaces.

The term “municipal solid waste (MSW)” means trash or garbage thrown away as everyday items from homes, school, hospitals and business. Municipal solid waste includes packaging, newspapers, clothing, appliances, and food rests. For the purposes of the present application the term “municipal solid waste” may be defined as a feedstock containing materials of items discarded by the public, such as mixed municipal waste given the waste code 200301 in the European Waste Catalog (EWC code 20 03 01).

The second syngas feed is rich in CO, for instance (by mole or volume, dry basis): 40-70% such as 60% CO, 1-10% CO₂ such as 5%, 20-40% H₂ such as 30% H₂, the balance being inerts: N₂+Ar, and H₂S. The high molar ratio of CO/CO₂ in the second syngas feed thus provides the same benefits of having a high CO/CO₂ molar ratio recited above.

The ATR-based syngas generated by reforming the stream rich in paraffins contains CO, CO₂ and H₂, has a composition which also ensures a CO/CO₂, needed for lower methanol catalyst volume and thereby, smaller MeOH loop. For instance, the composition of a first ATR-based syngas stream, which is the syngas withdrawn from the ATR in the reforming system is (by volume, dry basis): 40-70% H₂, 10-30% CO, 2-20% CO₂, 0.5-5% CH₄.

To obtain an optimized yield in the methanol production, the stoichiometry of H₂, CO and CO₂ needs to be considered. Hence, in an embodiment, the first or second syngas feed has a molar ratio CO/CO₂ greater than 1, such as greater than 2, e.g. 10 or higher. Suitably also, the first or second syngas feed has a module M=(H₂−CO₂)/(CO+CO₂) defined in terms of molar content, in the range 1.80-2.20, such as 1.95-2.10. Similarly, the ATR-based syngas, e.g. the first and second ATR-based syngas streams, may have a molar ratio CO/CO₂ greater than 1, such as greater than 2, e.g. 10 or higher. Suitably also, the ATR-based syngas has a module M=(H₂−CO₂)/(CO+CO₂) in the range 1.80-2.40, such as 1.95-2.10.

In an embodiment, the gasoline synthesis plant 200 comprises:
a reverse water gas shift (rWGS) unit, preferably an electrical rWGS unit (e-rWGS) unit, arranged to receive a portion of said first CO₂ rich feed 201 comprising CO₂ and a portion of said first H₂ rich feed 202 comprising H₂, to provide a rWGS syngas feed; and means such as a mixing unit or junction to combine the rWGS syngas feed with the remaining portion of: said first CO₂ rich feed 201 comprising CO₂ and said first H₂ rich feed 202 comprising H₂, and provide said second syngas feed 205. In a particular embodiment, the rWGS is also arranged to receive a portion of said by-product stream 242, 242′ rich in paraffins.

The rWGS reaction CO₂+H₂═CO+H₂O is endothermic, requiring a significant heat input. The rWGS unit is thus preferably electrically heated (e-rWGS unit). In that case, optionally a portion of the by-product stream rich in paraffins; optionally also a portion of the off-gas stream is sent to the e-rWGS unit, since the e-rWGS unit may also enable some steam reforming of these streams.

For details on e-rWGS, reference is given to e.g. applicant's WO 2022079098 (e-RWGS section therein).

In an embodiment, a methanol storage tank is arranged between said methanol synthesis unit 220 and said gasoline synthesis section 230, i.e. downstream the methanol synthesis unit and upstream the gasoline synthesis section, for storing at least a portion of the effluent stream 221 comprising methanol.

This provides a simple solution for coping with intermittent sources for producing the electricity required in e.g. upstream electrolysis. The methanol storage tank may be arranged downstream said water separation section for removing water. The water separation section is for instance a distillation column. The methanol storage tank accumulates the methanol from the overhead section of the distillation column, at for instance low pressure, such as less than 5 barg, for instance atmospheric pressure, thus enabling the use of inexpensive materials for such tank while also serving as buffer for any sudden variations in electricity due to the intermittent nature of the source producing it, such as wind and solar energy.

The plant (and process) according to the present invention thus enables not only improving hydrogen (H) and carbon (C) efficiency of the plant, while improving performance and thereby reducing the size of the methanol synthesis unit, for instance a MeOH-loop, or at least without changing the main equipment size in the methanol synthesis unit, but at the same time provides a robust plant which copes with the sudden and often huge variations in electricity supply for e.g. electrolysis of water or steam into the hydrogen required in the syngas feed for methanol production, or oxygen produced in the electrolysis which is used as oxidant stream in the ATR.

The first or second syngas feed as well as the ATR-based syngas may be combined and fed to the methanol synthesis unit.

Accordingly, in an embodiment, the at least a portion of said ATR-based syngas stream 41, 51, 53, 62, such as first, second or third ATR-based syngas stream 41, 51, 53 is arranged to be fed to the inlet of the methanol synthesis unit 220 in admixture with said CO₂ rich feed 201 and/or said H₂ rich feed 202, or in admixture with said first syngas feed 209, or in admixture with said second syngas feed 205.

In an embodiment, the methanol synthesis unit (220) is arranged for the ATR-based syngas stream 41, 51, 53, 62 being up to up to 50% by volume basis, such as 5-45%, for instance 15-45%, e.g. 10-40% or 20-40% of the inlet of the methanol synthesis unit 220.

Thereby, 5-50%, such as 10-40%; e.g. 15-30% of the effluent stream 221 comprising methanol, is obtained from the recycle streams, i.e. by-product stream rich in paraffins, optionally also off-gas stream(s). The ATR-based syngas stream may thus be 5, 10, 15, 20, 25, 30, 35, 40, 45, 50% volume basis of the inlet of the methanol synthesis unit.

The particular feeding point of the ATR-based syngas to the inlet of the methanol synthesis unit is, for instance, downstream the mixing point of said CO₂ rich feed and said H₂ rich feed and upstream a first syngas feed compressor arranged therein, thus in admixture with said first syngas feed, or in admixture with said second syngas feed. The H₂ rich feed from e.g. water electrolysis is provided by a dedicated H₂-compressor. The CO₂ rich feed, suitably after CO₂-gas cleaning, is combined with the H₂ rich feed from the dedicated H₂-compressor into said first syngas feed and then provided to the methanol reactor of the methanol synthesis unit by the first syngas feed compressor.

From e.g. water electrolysis, the hydrogen is compressed from about 1-5 bara (bar abs) to about 30 bara as the H₂-rich feed stream. The CO₂-rich feed is provided at about 30 bara, cleaned in the CO₂-gas cleaning, combined with the compressed H₂ rich feed steam and the ATR-based syngas stream, and then in said first syngas feed compressor being compressed to up to about 90 bara as the inlet to methanol synthesis unit. This is a more efficient approach to deliver the required pressure of up to about 90 bara than compressing the H₂-rich feed stream separately to inlet to methanol synthesis unit.

In another embodiment, the particular feeding point of the ATR-based syngas to the inlet of the methanol synthesis unit, may be together with the overhead recycle stream of the methanol synthesis unit, as recited below:

In an embodiment, the methanol synthesis unit is a methanol synthesis loop (MeOH loop), which comprises:
optionally, a cleaning section, such as a desulfurisation section, arranged to receive said CO₂ rich feed 201 and/or said H₂ rich feed 202, or said first syngas feed 209; or said second syngas feed 205, thereby providing a cleaned methanol syngas feed, such as a desulfurized methanol syngas feed;
a methanol reactor arranged to receive the cleaned methanol syngas feed, such as the desulfurized methanol syngas feed, and to produce a raw methanol effluent stream;
a first separator arranged to receive the raw methanol effluent stream, and to produce i.e. to provide a bottom stream as said effluent stream 221 comprising methanol, suitably after being fed to a second separator such as low-pressure separator from which an off-gas is generated, and said first separator also being arranged to produce an overhead recycle stream;
a recycle compressor arranged to recycle the overhead recycle stream to the methanol reactor.

In an embodiment, the methanol synthesis unit further comprises:
means such as a mixing unit or junction to combine the overhead recycle stream with: said CO₂ rich feed 201 and/or said H₂ rich feed 202, or said first syngas feed 209, or said second syngas feed (205); i.e. the overhead recycle is provided in admixture with any of the above streams; preferably, wherein any of the above streams is provided as said cleaned methanol syngas feed at said mixing point or junction it would be understood that the term “junction” may be used interchangeably with the term “juncture”. It denotes a mixing point.

In an embodiment, the methanol synthesis unit (220), i.e. said MeOH loop, may further comprise:
means such as a mixing unit or junction, suitably located downstream said recycle compressor, to combine the ATR-based syngas stream 41, 52, 53, 62, such as the first, second or third ATR-based syngas 41, 51, 53 stream, with the overhead recycle stream.

Accordingly, in an embodiment, the methanol synthesis unit 220 further comprises:
means such as a mixing unit or junction, suitably located downstream said recycle compressor, to combine the ATR-based syngas stream 41, 52, 53, 62 with the overhead recycle stream prior to being fed to the inlet of the methanol synthesis unit 220 in admixture with said CO₂ rich feed 201 and/or said H₂ rich feed 202, or in admixture with said first syngas feed 209, or in admixture with said second syngas feed 205; preferably in admixture with said cleaned methanol syngas feed, such as a desulfurized methanol syngas feed.

Upstream the methanol reactor, as recited above, suitably as part of the methanol synthesis unit, there may be provided a cleaning section such as a desulfurisation section, for instance as a sulfur absorber and sulfur guard, to remove sulfur from a syngas feed, here to remove any sulfur in, for instance, the first syngas feed, since sulfur is detrimental for the downstream methanol reactor catalyst. By combining the ATR-based syngas with the overhead recycle instead of with e.g. the first syngas feed, the volumetric flow to the desulfurisation section remains unchanged, thus avoiding the penalty of increasing the sulfur removal capacity by providing a correspondingly larger desulfurisation section due to addition of ATR-based syngas. After being combined with the overhead recycle, the ATR-based syngas is then provided in admixture with said CO₂ rich feed and/or said H₂ rich feed, or in admixture with said first syngas feed, or in admixture with said second syngas feed.

From the overhead recycle stream of the methanol synthesis unit, suitably upstream the recycle compressor, an optional fuel gas stream may be withdrawn from which a hydrogen stream is recovered. This hydrogen stream is herein referred to as excess hydrogen stream from the methanol synthesis unit; and is for instance a hydrogen stream of a hydrogen recovery unit such as a pressure swing adsorption unit (PSA unit), or a hydrogen stream of purge gas scrubber suitably arranged upstream the hydrogen recovery unit, e.g. PSA-unit. Thereby, additional hydrogen is internally produced in the plant or process. The hydrogen stream of the hydrogen recovery unit, e.g. PSA unit, is suitably sent to e.g. the hydroisomerisation (HDI) reactor and/or hydrocracking (HCR) reactor of the upgrading section of the plant. The hydrogen stream of the purge gas scrubber is suitably sent to the hydrogenation section of the reforming system of the plant. The hydrogenation section serves i.a. to remove any olefins being fed to the reforming system.

Accordingly, in an embodiment, the methanol synthesis unit further comprises: a conduit for withdrawing a portion of said overhead recycle stream as a fuel gas, suitably upstream the recycle compressor, and providing therefrom said excess hydrogen stream from the methanol synthesis unit; preferably, wherein said excess hydrogen stream is provided by the methanol synthesis unit further comprising a hydrogen recovery unit arranged to receive said fuel gas and provide a hydrogen-rich stream as said excess hydrogen stream 255, the hydrogen recovery unit being at least one of: a gas separator such as a gas scrubber, a membrane unit, and a pressure swing adsorption (PSA) unit.

It would be understood that in connection with this embodiment, the term “fuel gas” is used interchangeably with the term “purge gas”.

In an embodiment, as already recited the methanol synthesis unit 220 is arranged to provide an excess hydrogen stream, i.e. excess hydrogen stream 255 from the methanol synthesis unit 220; the plant 200 further comprises in said reforming system 100 a hydrogenation section 10, and said hydrogenation section is arranged to receive said excess hydrogen stream 255 from the methanol synthesis unit 220.

The provision of the excess hydrogen stream from the methanol synthesis unit enables a simpler layout in the reforming system by eliminating the need e.g. a hydrogen recovery section within the reforming system of the plant to provide a hydrogen rich stream and thereby also a hydrogen compressor to send the hydrogen rich stream to the hydrogenation section of the reforming system.

In an embodiment, the by-product stream 242, 242′ rich in paraffins, is a stream rich in propane and/or butane, such as a LPG stream.

The gasoline synthesis section 230 comprises a methanol-to-gasoline section (MTG section), i.e. MTG-loop, and a downstream distillation section comprising a de-ethanizer and a LPG-splitter; so the LPG-stream is for instance withdrawn from the LPG splitter.

The term “rich in propane and/or butane” means that at least 50%, such as at least 60%, preferably at least 75% of this first stream is propane and/or butane. Typically, LPG contains 70-80 vol % butane, 20-30 vol % propane and some other hydrocarbons. Hence, in an embodiment, the first stream rich in paraffins is an LPG stream (LPG feed). Propylene, butylenes and various other hydrocarbons are usually also present in LPG in small concentrations such as C₂H₆, CH₄ etc. An LPG feed may also comprise olefins.

In an embodiment, the by-product stream 242, 242′ rich in paraffins, is a naphtha stream. As used herein, “naphtha stream” means C5-C9 hydrocarbons boiling in the range 30-160° C., such as C5-C8 hydrocarbons.

In an embodiment, the plant 200 is arranged to provide one or more off-gas streams 253, 253′, said one or more off-gas streams 253, 253′ being a waste-gas stream rich CO₂, H₂, CH₄, and said reforming system 100 is arranged to receive at least a portion of said one or more off-gas stream(s) 253, 253′.

For the purposes of the present application, an off-gas stream is regarded separately from a by-product stream rich in paraffins. The latter is regarded as a by-product stream, the former as a waste gas stream.

The off-gas stream(s) are streams comprising CO₂, H₂, CH₄, and higher hydrocarbons which are also produced in the gasoline synthesis plant. For instance, an off-gas stream may come from the so-called methanol-to-gasoline section (MTG-section) of the gasoline synthesis section (230), as it will become apparent farther below. Other off-gas streams may come from the upgrading section of the gasoline synthesis plant. The off-gas streams often have no efficient use, apart from using them in fired equipment, such as in fired heaters, which causes CO₂ emission. These off-gas streams are now recycled as part of the gasoline synthesis process itself, in order to improve overall C-efficiency of this process. Furthermore, recycling propane and/or butane rich stream and/or off-gas stream via autothermal reforming, in a CO₂ and H₂ feed based methanol synthesis enhances methanol loop performance, as explained previously.

For instance, the off-gas stream is from the first separator arranged to receive the raw methanol effluent stream recited above. Hence, in the methanol synthesis unit, the first separator arranged to receive the raw methanol effluent stream, and to produce the overhead recycle stream, as well as a bottom stream as said effluent stream (221) comprising methanol after being fed to a second separator such as low-pressure separator from which the off-gas is generated.

For instance, the off-gas stream is produced from the overhead recycle stream. Hence, as already described, from the overhead recycle stream, suitably upstream the recycle compressor, a fuel gas (purge gas) stream is withdrawn from which a hydrogen stream is recovered as said excess hydrogen stream from the methanol synthesis unit, for instance in a hydrogen recovery unit such as a pressure swing adsorption unit (PSA unit). The hydrogen recovery unit produces also a waste gas, such as a PSA-waste gas, as said off-gas stream.

For instance, the off-gas stream is a stream comprising light-end hydrocarbons from a fractionation unit, e.g. a distillation unit, in the upgrading section of the gasoline synthesis plant.

Hence, one or more additional off-streams in the plant are arranged to be fed to the reforming system, optionally in combination with said by-product stream rich in paraffins.

In the reforming system, the reforming unit is an ATR. The ATR is thus arranged to receive the first stream along with the oxidant stream which includes oxygen from the electrolysis section of the gasoline synthesis plant, and carry out an autothermal reforming step, to provide e.g. said first ATR-based syngas stream.

Use of an ATR in this manner allows recycling of e.g. the LPG streams and/or off-gas streams, such that additional CO₂ emissions can be avoided, or significantly minimised. The main elements of an ATR reactor are a burner, a combustion chamber, and a catalyst bed contained within a refractory lined pressure shell. In an ATR reactor, partial oxidation or combustion of a hydrocarbon feed by sub-stoichiometric amounts of oxygen is followed by steam reforming of the partially combusted hydrocarbon feed stream in a fixed bed of steam reforming catalyst. Steam reforming also takes place to some extent in the combustion chamber due to the high temperature. The steam reforming reaction is accompanied by the water gas shift reaction. Typically, the gas is at or close to equilibrium at the outlet of the ATR reactor with respect to steam reforming and water gas shift reactions. The temperature of the exit gas is typically in the range between 85° and 1100° C. More details of ATR and a full description can be found in the art such as “Studies in Surface Science and Catalysis, Vol. 152,” Synthesis gas production for FT synthesis “; Chapter 4, p. 258-352, 2004”.

Suitable process conditions (temperatures, pressures, flow rates etc.) and suitable catalysts for such steam reforming processes are known in the art.

In an embodiment, the plant 200 further comprises in said reforming system 100 a separation section 50, arranged to receive at least a portion of said ATR-based syngas stream as a first ATR-based syngas stream 41 and separate it into at least a second ATR-based syngas stream 51 and a process condensate 52. This separation section advantageously removes water from the first ATR-based syngas stream, which is detrimental for its use in methanol synthesis.

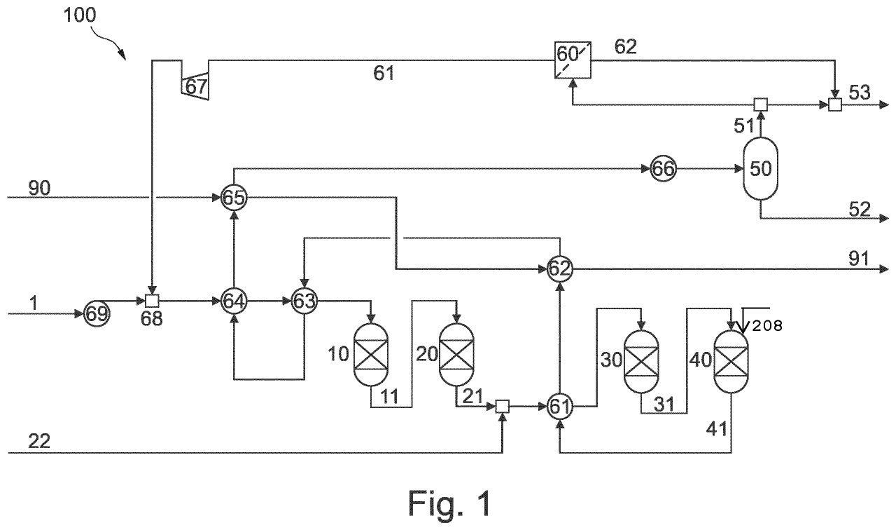

The reforming system may comprise said hydrogenation section, which is arranged to receive the first reforming feed stream, and provide a hydrogenated first reforming feed stream. In the hydrogenation section, the first reforming feed stream is mixed with hydrogen feed, suitably an excess hydrogen stream from the methanol synthesis unit of the plant, as earlier recited, and passed over a catalyst active in hydrogenation. Again, the provision of excess hydrogen stream from the methanol synthesis unit enables a simpler layout without need of a hydrogen recovery section in the ATR-based syngas to provide a hydrogen rich stream and thereby a hydrogen compressor to send the hydrogen rich stream to the hydrogenation section, as e.g. illustrated in appended FIG. 1. The hydrogenation section may comprise one or more hydrogenation reactors in series. Hydrogenation converts unsaturated hydrocarbon components, such as olefins, e.g. propylene or butylene, to the corresponding saturated hydrocarbons, which can reduce or avoid carbon formation (in a reforming step) by converting olefins into alkanes. Hydrogenation catalysts and reactors suitable for such processes are commercially available and known to the skilled person.

The reforming system may also comprise a desulfurisation section arranged to receive the hydrogenated first reforming feed stream, and provide a desulfurised first and/or second stream. Typically, the desulfurisation section comprises one or more hydrodesulfurization (HDS) reactors. Desulfurisation converts sulfur-containing compounds in the first reforming feed stream to hydrocarbons (typically saturated hydrocarbons) and sulfur-containing compounds (e.g., H₂S) as by-product. This can reduce catalyst poisoning in subsequent conversion steps. Desulfurisation catalysts and reactors suitable for such processes are commercially available and known to the skilled person. Substances other than sulfur that might need to be removed in such a purification step include chlorine, dust and heavy metals.

A pre-reforming section, i.e. a pre-reformer (or interchangeably, a pre-reforming unit), such as one or more pre-reformers, may be arranged to receive the first stream and carry out a pre-reforming step. A pre-reformed stream is provided. Pre-reforming is an additional reforming step, which allows a syngas with a desired composition to ultimately be obtained, i.e. in which higher hydrocarbons are converted to methane. Pre-reforming suitably takes place at ca. 350-700° C. to convert higher hydrocarbons as an initial step. Pre-reforming catalysts and reactors suitable for such processes are commercially available and known to the skilled person. Pre-reformer units used in the present invention are catalyst-containing reactor vessels, and are typically adiabatic. In the pre-reforming units, heavier hydrocarbon components in the hydrocarbon feedstock are steam reformed and the products of the heavier hydrocarbon reforming are methanated. The skilled person can construct and operate suitable pre-reforming units as required. Pre-reforming units suitable for use in the present system/process are provided in applicant's co-pending applications EP20201822 and EP21153815. The pre-reformed stream comprises methane, hydrogen, carbon monoxide and also carbon dioxide. The pre-reformed stream at the outlet of the pre-reformer may be in the temperature range: 400° C.-500° C.

As the first ATR-based syngas stream is at an elevated temperature (e.g. about 1100° C.) at the outlet of the ATR, it can advantageously be heat-exchanged with upstream components in the system, for effective energy use in the reforming system. The reforming system may therefore comprise one or more heat exchangers, being arranged to provide heat exchange between the first ATR-based syngas stream and one or more of: the first reforming feed stream, the desulfurised first reforming feed stream and boiler feed water stream. Suitably, the first ATR-based syngas stream is heat exchanged with the desulfurised first reforming feed stream first, then a boiler feed water stream, and then with the first reforming feed stream. Alternatively, or additionally, one or more electrical heaters may be used to raise the temperature of one or more of: the first reforming feed stream, the hydrogenated first reforming feed stream, the desulfurised first reforming feed stream and boiler feed water stream.

As explained above, the reforming system may further comprise a second reforming feed stream being an off-gas stream comprising CO₂, H₂ and CH₄, said second stream being arranged to be mixed with the first reforming feed stream, upstream the inlet of the ATR.

In an embodiment, the reforming system comprises a hydrogen recovery section 60, said hydrogen recovery section 60 being arranged to receive at least a portion of the second ATR-based syngas stream 51 and provide at least a hydrogen-rich stream 61 and a fourth ATR-based syngas stream 62; and at least a portion of the second ATR-based syngas stream 51 and at least a portion of the fourth ATR-based syngas stream 62 are arranged to be combined into third ATR-based syngas stream 53. The hydrogen recovery section may comprise a membrane hydrogen separation unit or a PSA (pressure swing adsorption) unit or both.

At least a portion of the hydrogen-rich stream obtained from the hydrogen recovery section and/or a portion of the second ATR-based syngas stream from the separation section may be used in the hydrogenation section of the reforming system. Therefore, at least a portion of the hydrogen-rich stream may be combined with e.g. the first reforming feed upstream the hydrogenation section. Optionally, recovered H₂ can also be used in the hydroisomerisation reactor (HDI reactor) of the upgrading section of the plant. See farther below.

Further integration and improved hydrogen efficiency—by internal sourcing of hydrogen—of the gasoline synthesis plant is thereby also achieved.

In an embodiment, as recited farther above, the methanol synthesis unit 220 is arranged to provide an excess hydrogen stream, i.e. excess hydrogen stream from the methanol synthesis unit 220; the gasoline synthesis section 230 comprises: a methanol-to-gasoline section (MTG section), i.e. MTG-loop, and a downstream distillation section comprising a de-ethanizer and a LPG-splitter; the upgrading section 240 comprises: a hydroisomerisation (HDI) reactor, optionally a hydrocracking (HCR) reactor; and said HDI reactor and/or HCR reactor is arranged to receive: a portion of the first or second H₂ rich feed 202, 202′ comprising H₂, and/or said excess hydrogen stream from the methanol synthesis unit 220; optionally, a portion of the hydrogen-rich stream 61 from hydrogen recovery section 60 of the reforming system (100).

Thereby, hydrogen required for the HDI and/or HCR reactors in the upgrading section is also sourced internally, rather than costly external sourcing from outside the battery limits (BL) of the plant. As already described, the excess hydrogen stream from the methanol synthesis unit is suitably generated from the overhead recycle stream, for instance by providing said hydrogen recovery unit, such as a pressure swing adsorption (PSA) unit arranged to receive a portion of the overhead recycle stream and provide said excess hydrogen stream.

As is well-known in the art, gasoline synthesis from oxygenates such as methanol involves plants comprising a MTG section (methanol-to-gasoline section) and a downstream distillation section. The MTG section may also be referred as MTG loop and comprises: a MTG reactor; product separator for withdrawing a bottom water stream, an overhead recycle stream from which an optional fuel gas stream may be withdrawn, as well as a raw gasoline stream comprising C2 compounds, C3-C4 paraffins (LPG) and C5+ hydrocarbons (gasoline boiling components); and a recycle compressor for recycling the overhead recycle stream by combining it with the oxygenate feed stream, e.g. methanol feed stream. The overhead recycle stream (or simply, recycle stream) acts as diluent, thereby reducing the exothermicity of the methanol conversion in the MTG reactor. In the distillation section, C2 compounds are removed in a de-ethanizer, such as de-ethanizer column, and then a C3-C4 fraction is removed as LPG as the overhead stream in a LPG-splitting column (LPG splitter), while stabilized gasoline is withdrawn as the bottoms product. The stabilized gasoline or the heavier components of the stabilized gasoline, such as the C9-C11 fraction, may optionally be further treated and thereby refined in downstream upgrading section, by e.g. conducting hydroisomerisation (HDI) into an upgraded gasoline product as the gasoline product stream.

The invention provides also process for gasoline synthesis of a first CO₂ rich feed 201 comprising CO₂, and a first H₂ rich feed 202 comprising H₂, or from a first syngas feed 209 which combines said first CO₂ rich feed and said first H₂ rich feed; or from a second syngas feed (205) comprising a carbon oxide and hydrogen, said process comprising the steps of:
providing a gasoline synthesis plant 200 according to any one of the preceding embodiments;
supplying CO₂ rich feed 201 and H₂ rich feed 202, or said first syngas feed 209, or said second syngas feed 205, to the methanol synthesis unit 220, and providing an effluent stream 221 comprising methanol;
supplying at least a portion of the effluent stream 221 comprising methanol from the methanol synthesis unit 220 to the gasoline synthesis section 230, and providing a raw product 231 containing hydrocarbons boiling in the gasoline range;
supplying at least a portion of the raw product 231 from the gasoline synthesis section 230 to optional upgrading section 240, and providing a gasoline product stream 241; said optional upgrading section 240 comprising: hydroisomerisation (HDI) reactor, optionally hydrocracking (HCR) reactor, thereby providing said gasoline product stream 241;
withdrawing from to the gasoline section 230 and/or the optional upgrading section 240 a first stream 242, 242′ rich in paraffins; optionally one or more off-gas streams 253, 253′, such as a second stream 253 from the methanol synthesis unit 220 and/or second stream 253′ from the optional upgrading section 240;
supplying at least a portion of a first stream 1 as said first stream 242, 242′ rich in paraffins, optionally at least a portion of said one or more off-gas streams 253, 253′, to reforming system 100, performing autothermal reforming step in the ATR 40, and providing an ATR-based syngas stream 41, 51, 53, 62, such as a first, second or third ATR-based syngas stream 41, 51, 53;
supplying at least a portion of said ATR-based syngas stream 41, 51, 53, 62 to the inlet of the methanol synthesis unit 220, preferably in admixture with said CO₂ rich feed 201 and/or said H₂ rich feed 202, or in admixture with said first syngas feed 209, or in admixture with said second syngas feed 205;
wherein the process does not comprise a primary reforming step in connection with said autothermal reforming step, such as a steam methane reforming in a SMR, (i.e. other than pre-reforming, only ATR) prior to the autothermal reforming step;
wherein the process does not comprise steam reforming of a hydrocarbon feed gas for providing said first or second syngas feed 205.

As in connection with the plant (gasoline synthesis plant), the process does not comprise a primary reforming step in connection with said autothermal reforming step, such as a steam methane reforming (SMR) prior to the autothermal reforming step; or convection heated reforming such as in a heat exchange reformer operating in parallel or in series with the autothermal reforming. Hence, again, other than pre-reforming, there is only ATR in the reforming system 100.

In an embodiment,
excess hydrogen stream 255 from the methanol synthesis unit 220, such as a portion of said excess hydrogen stream 255 from the methanol synthesis unit 220, is supplied to the optional upgrading section 240, preferably to said HDI reactor optional HCR reactor.
and/or
the methanol synthesis unit 220 provides an excess hydrogen stream 255, wherein said excess hydrogen stream 255 from the methanol synthesis unit 220, such as a portion of said excess hydrogen stream 255 from the methanol synthesis unit 220, is supplied to the reforming system 100; preferably, said reforming system 100 comprising a hydrogenation section 10, and wherein said excess hydrogen stream 255 is supplied to said hydrogenation section 10.

In an embodiment, the gasoline synthesis section 230 comprises: a methanol-to-gasoline section (MTG section), and a downstream distillation section comprising a de-ethanizer and a LPG-splitter;
said HDI reactor and/or optional HCR reactor of said optional upgrading section 240, and: a portion of the first or second H₂ rich feed 202, 202′ comprising H₂, and/or an excess hydrogen stream 255 from the methanol synthesis unit (220), such as a portion of said excess hydrogen stream 255 from the methanol synthesis unit (220), is suppled to said HDI reactor and/or optional HCR reactor.

In an embodiment, said first reforming feed 1, 242, 242′ is less than 15 wt % of said raw product 231 containing hydrocarbons boiling in the gasoline range, or less than 15 wt % of said gasoline product stream 241.

In an embodiment, the process further comprises:
an electrolysis step of water feedstock 203 to provide second H₂ rich feed 202′ comprising H₂, optionally as said first H₂ rich feed 202 comprising H₂; said electrolysis step also providing a first oxygen stream 206, combining steam 207 with the oxygen stream to provide an oxidant stream 208, and supplying the oxidant stream 208 to the autothermal reforming step;
and/or an electrolysis step of a second CO₂ rich feed 201′ comprising CO₂ to provide a CO-enriched feed 204; and combining the second H₂ rich feed 202′ comprising H₂ and the CO-enriched feed 204 to provide said second syngas feed 205; said electrolysis step also providing a second oxygen stream 206′; optionally combining said second oxygen stream 206′ with said first oxygen stream 206 and said steam 207;
or
a thermal decomposition step, such as gasification, of a biomass feedstock for providing a crude syngas feed, and a purification step of the crude syngas feed for providing said second syngas feed 205;
or
a reverse water gas shift (rWGS) step, preferably in an electrical rWGS (e-rWGS) unit, of a portion of said first CO₂ rich feed 201 comprising CO₂ and a portion of said first H₂ rich feed 202 comprising H₂, to provide a rWGS syngas feed; and then combining the rWGS syngas feed with the remaining portion of: said first CO₂ rich feed 201 comprising CO₂, and said first H₂ rich feed 202 comprising H₂, to provide said second syngas feed 205; and optionally, supplying a portion of said by-product stream 242, 242′ rich in paraffins to the rWGS unit.

In an embodiment, steam stream 207 is combined with the first oxygen stream 206 to provide an oxidant stream 208; and said oxidant stream 208 is supplied to the ATR 40 of the reforming system 100;
optionally, the first 206 and/or second 206′ oxygen streams, with said steam stream 207 are combined to provide oxidant stream 208.

In an embodiment, the process further comprises supplying at least a portion of the effluent stream 221 comprising methanol to a methanol storage tank, said methanol storage tank being provided between said methanol synthesis unit 220 and said MTJ synthesis section 230.

In an embodiment, the process comprises feeding the ATR-based syngas to the inlet of the methanol synthesis unit is at a feeding point which is downstream the mixing point of said CO₂ rich feed and said H₂ rich feed and upstream first syngas feed compressor arranged therein, thus in admixture with said first syngas feed, or in admixture with said second syngas feed. The H₂ rich feed from e.g. water electrolysis is provided by a dedicated H₂-compressor. The CO₂ rich feed, suitably after CO₂-gas cleaning, is combined with the H₂ rich feed from the dedicated H₂-compressor into said first syngas feed and then provided to the methanol reactor of the methanol synthesis unit by the first syngas feed compressor.

In an embodiment, the ATR-based syngas stream 41, 51, 53, 62 is up to 50% by volume basis, such as 5-45%, for instance 15-45%, e.g. 10-40% or 20-40% of the inlet of the methanol synthesis unit 220.

The feeding point of the ATR-based syngas to the inlet of the methanol synthesis unit, may be together with the overhead recycle stream of the methanol synthesis unit. Hence, in an embodiment, the methanol synthesis unit is a methanol synthesis loop. Accordingly, in an embodiment, the methanol synthesis unit 220 of the gasoline synthesis plant 200 is provided with a methanol reactor; and the step of supplying at least a portion of said ATR-based syngas stream 41, 51, 53, 62, such as first, second or third ATR-based syngas stream 41, 51, 53, to the inlet of the methanol synthesis unit 220, comprises:
withdrawing a raw methanol effluent stream from the methanol reactor;
separating from said raw methanol effluent stream an overhead recycle stream and directing it to the methanol reactor; and
providing the at least a portion of the ATR-based syngas stream 41, 51, 53, 62, such as first, second or third ATR-based syngas 41, 51, 53, to said overhead recycle stream;
thereby supplying said ATR-based syngas stream to said inlet of the methanol synthesis unit 220.

Optionally, said CO₂ rich feed 201 and/or said H₂ rich feed 202, or said first syngas feed 209; or said second syngas feed 205, is supplied to a cleaning section, such as a desulfurisation section, arranged to receive said CO₂ rich feed 201 and/or said H₂ rich feed 202, or said first syngas feed; or said second syngas feed (205), thereby providing a cleaned methanol syngas feed, such as a desulfurized methanol syngas feed;

In an embodiment, the process comprises combining the ATR-based syngas stream 41, 52, 53, 62 with the overhead recycle stream prior to being fed to the inlet of the methanol synthesis unit 220 in admixture with said CO₂ rich feed 201 and/or said H₂ rich feed 202, or in admixture with said first syngas feed, or in admixture with said second 205 syngas feed; preferably in admixture with said cleaned methanol syngas feed, such as a desulfurized methanol syngas feed.

In an embodiment, the process comprises withdrawing a portion of said overhead recycle stream as a fuel gas, suitably upstream a recycle compressor for recycling of said overhead recycle stream, and providing therefrom said excess hydrogen stream 255 from the methanol synthesis unit 220; preferably, wherein said excess hydrogen stream 255 is provided by the methanol synthesis unit 220 further comprising supplying said fuel gas a hydrogen recovery unit arranged to receive said fuel gas and provide a hydrogen-rich stream as said excess hydrogen stream 255, in which the hydrogen recovery unit is at least one of: a gas separator such as a gas scrubber, a membrane unit, and a hydrogen recovery unit such as pressure swing adsorption (PSA) unit.

In an embodiment, the by-product stream 242, 242′ rich in paraffins, is a stream rich in propane and/or butane, such as a LPG stream; and/or a naphtha stream.

In an embodiment, said one or more off-gas streams 253, 253′ is a waste-gas stream rich in CO₂, H₂, CH₄.

In an embodiment, the process further comprises:
supplying in reforming system 100 at least a portion of said first-based syngas stream 4), i.e. from the ATR 40, to separation section 50, and separating it therein into at least second-based syngas stream 51 and process condensate 52.

In an embodiment, the step of supplying at least a portion of said first stream 1, 242, 242′ rich in paraffins, optionally at least a portion of said one or more off-gas streams 253, 253′, to said reforming system 100, and providing a first, second or third ATR-based syngas stream 41, 51, 53, comprises:
optionally, hydrogenating the first reforming feed stream 1 in the hydrogenation section 10 to provide a hydrogenated first reforming feed stream 11;
optionally, desulfurising said hydrogenated first reforming feed stream 11 in desulfurisation section 20, to provide a desulfurised first reforming feed stream 21;
pre-reforming the first reforming feed stream 21 in pre-reforming section 30, to provide a pre-reformed first reforming feed stream 31;
performing said autothermal reforming step on said first reforming feed stream 1, 11, 21, 31 to provide said first ATR-based syngas stream 41.

As recited, the process, in an embodiment, comprises: a methanol-to-gasoline step (MTG step) in the MTG section i.e. MTG-loop of the gasoline synthesis section 230, a subsequent distillation in a de-ethanizer and LPG-splitter in the distillation section of the gasoline synthesis section 230, thereby providing the raw product 231; and subsequent upgrading of the raw product 231 in a hydroisomerization (HDI) step in the HDI reactor of the optional upgrading section 240, optionally a hydrocracking (HCR) step in the HCR reactor, thereby providing the gasoline product stream (241);

the process further comprising supplying a portion of the first H₂ rich feed 202 comprising H₂ to said HDI or HCR step, and/or supplying an excess hydrogen stream from the methanol synthesis unit, such as a portion of said excess hydrogen stream from the methanol synthesis unit 220, optionally also supplying a portion of a hydrogen-rich stream 61 from hydrogen recovery section (60) of reforming system to said HDI or HCR step.

In an embodiment, the step of supplying at least a portion of the raw product 231 from the gasoline synthesis section 230 to upgrading section (240), is optional.

It would be understood that any of the embodiments and associated benefits in connection with the gasoline synthesis plant, are applicable to the process, and thus may be used in connection with the corresponding embodiments of the process; or vice versa.

### SPECIFIC EMBODIMENTS

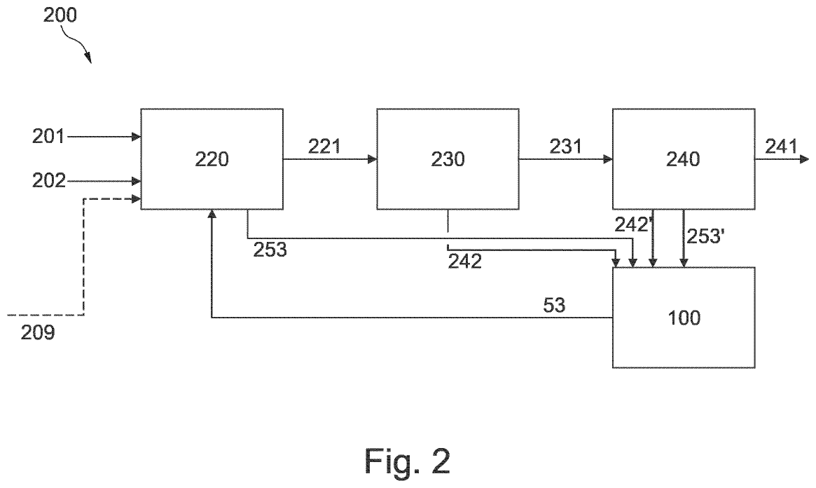

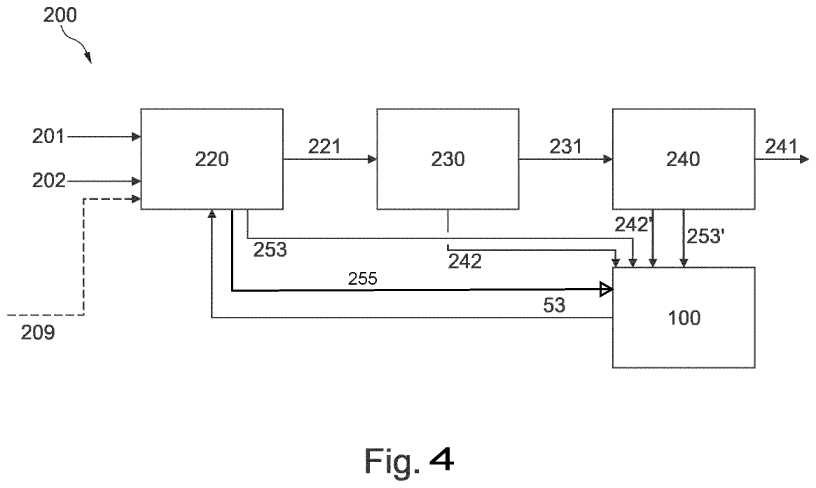

FIG. 1 shows a layout of the reforming system 100. A first stream rich in paraffins 1, such an LPG feed, corresponding to first stream 242, 242′ in FIG. 2, is compressed in first pump 69. The compressed first reforming feed stream 1 is—in this layout—mixed with hydrogen rich stream 61 at mixer 68 before being passed through heat exchangers 64, 63 to heat exchange with the first ATR-based syngas stream 41 from downstream autothermal reformer (ATR, 40) under the addition of oxidant stream 208. In another layout (not shown), the hydrogen rich stream to mixer 68 is an excess hydrogen stream 255 from the methanol synthesis unit 220 (see also FIG. 4). The heated first reforming feed stream is hydrogenated in hydrogenation section 10 to provide a hydrogenated first reforming feed stream 11 which is subsequently desulfurised in desulfurisation section 20, to provide a desulfurised first reforming feedstream 21. This desulfurised first reforming feed stream 21 may be mixed with process steam 22, and the mixed stream is again heat exchanged with the first ATR-based syngas stream 41. The desulfurised first reforming feed stream 21 is pre-reformed in pre-reforming section 30, to provide a pre-reformed stream 31. An autothermal reforming (ATR) step is performed on the pre-reformed stream 31 in ATR 40, to provide an ATR-based stream, such as a first ATR-based syngas stream 41. First ATR-based syngas stream 41 is then heat exchanged with boiler feed water 90 in waste heat boiler 62, providing export steam 91. Subsequently, first ATR-based syngas stream 41 is passed through heat exchangers 64, 63 (as noted above), and then heat-exchanged once more with boiler feed water 90 in heat exchanger 65. Additional cooling takes place in cooling unit 66. The first ATR-based syngas stream 41 is passed to a separation section 50 where it is separated into at least a second ATR-based syngas stream 51 and a process condensate 52. A portion of the second ATR-based syngas stream 51 is passed to hydrogen recovery section 60, where a hydrogen-rich stream 61 is separated and a fourth ATR-based syngas stream 62 is provided. A portion of the second ATR-based syngas stream 51 and a portion of the fourth ATR-based syngas stream 62 are combined into third ATR-based syngas stream 53. The hydrogen-rich stream 61 is compressed at compressor 67, and then combined with the first reforming feed stream 1, upstream the hydrogenation section 10 (as noted above).

Overall, in the illustrated system of FIG. 1, by-product stream rich in paraffins, optionally also an off-gas stream, to the reforming system are hydrogenated, desulfurized and prereformed before sending it to the ATR. The effluent stream from the ATR (first ATR-based syngas stream) gets cooled in series of heat exchangers by pre-reformer feed preheat, steam generation in waste heat boiler, feed preheater, first feed vaporizer, preheating of boiler feed water etc. The water in the effluent stream gets condensed and then separated, thereby providing a second ATR-based syngas stream. A part of ATR-based syngas is then used for H₂ recovery for internal use for hydrogenation and pre-reforming, or in a HDI reactor in the upgrading section of the gasoline plant. The rest of the ATR-based syngas is sent as a third ATR-based syngas stream to the methanol synthesis unit, as illustrated in FIG. 2.

FIG. 2 shows a gasoline synthesis plant 200 according to the invention. A reforming system 100, as per FIG. 1 is provided to make the recycling of the by-product stream rich in paraffins 1, 242, 242′ possible. It would thus be understood, that in the plant 200 of FIG. 2, stream 242, 242′ corresponds to stream 1 in FIG. 1. A first CO₂ rich feed 201 comprising CO₂ and a first H₂ rich feed 202 comprising H₂, suitably after combining them into a first syngas feed 209, are sent to methanol synthesis unit 220 (methanol loop, 220), from which an effluent stream 221 comprising methanol is provided. From the methanol synthesis unit 220 an off-gas stream 253, suitably from a product separator arranged therein (not shown) is generated and fed to the reforming system 100, as so is another off-gas stream 253′ from the upgrading section 240. The effluent stream comprising methanol 221 is supplied to gasoline synthesis section 230 comprising (not shown in the figure): MTG section (methanol-to-gasoline section) and a downstream distillation section. The MTG section is also referred as MTG loop and comprises: a MTG reactor; product separator for withdrawing a bottom water stream, an overhead recycle stream from which an optional fuel gas stream may be withdrawn, as well as a raw gasoline stream; and a recycle compressor for recycling the overhead recycle stream by combining it with the effluent stream 221 comprising methanol, i.e. methanol feed stream. From the fuel gas stream, off-gas stream may be generated. In the distillation section, C2 compounds are removed in a de-ethanizer, such as de-ethanizer column, and then a C3-C4 fraction overhead stream is removed as an LPG stream, hence suitably as first stream 242, in a LPG splitter, while stabilized gasoline as the raw product 231 is withdrawn as the bottoms product therefrom. The raw product 231 is optionally further treated and thereby refined in downstream upgrading section 240, e.g. by conducting hydroisomerisation (HDI) in a HDI reactor (not shown) into an upgraded gasoline product as the gasoline product stream 241. Hydrocracking (HCR) may also be conducted in a HCR reactor (not shown) in the upgrading section (240). An off-gas stream 253′ may also be generated. The by-product stream rich in paraffins 242, 242′ is fed to a system 100 as described above, and a third ATR-based syngas stream, such as ATR-based stream 53 is provided, which is then recycled to the methanol synthesis unit 220. From the reforming system 100, the first or second ATR-based syngas stream 41, 51 may also be recycled to the methanol synthesis unit 220 (not shown).

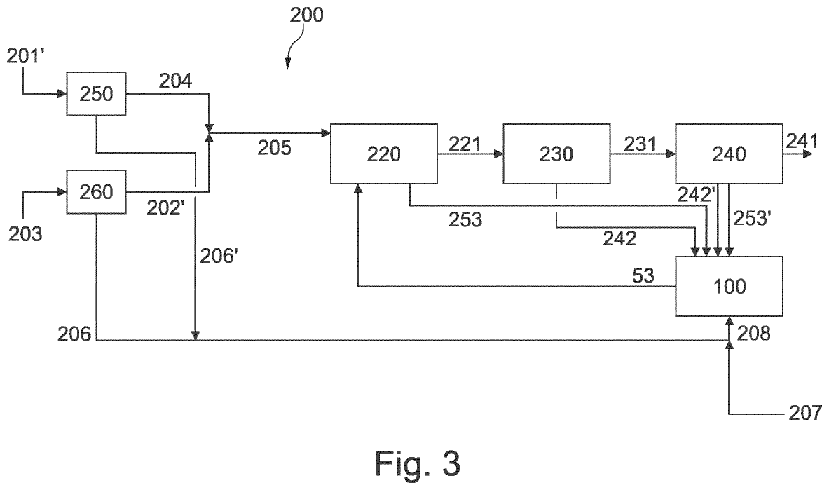

The ATR-based syngas 41, 51, 53 is a minor syngas stream to the gasoline synthesis plant 200, more specifically to the methanol synthesis unit 220, compared to the major syngas stream thereto, namely the first syngas feed (resulting from combining the first CO₂ rich feed 201 and the first H₂ rich feed 202; or compared the second syngas feed resulting from combining electrolysis of CO₂ and water/steam, as illustrated in FIG. 3, or by the thermal decomposition of a biomass feedstock, i.e. a solid renewable feed, or from rWGS of the first CO₂ rich feed and first H₂ rich feed. For instance, the ATR-based syngas 41, 51, 52 is less than 20% (volume basis), for instance in the range 10-15%, of the first syngas feed. Compared to the prior art, where a reforming system is provided in the main syngas stream to the gasoline synthesis plant, the present invention enables a much smaller reforming system 100, with significantly reduced plot size and attendant capital and operating expenses,

FIG. 3 shows a gasoline synthesis plant 200 as in FIG. 2, in which the methanol synthesis unit 220 is now fed with second syngas feed 205. The plant 200 comprises an electrolysis unit 260 arranged to receive a water feedstock 203, i.e. steam and/or water, and provide a second H₂ rich feed 202′ comprising H₂; optionally as said first H₂ rich feed 202, as well as first oxygen stream 206. The plant 200 further comprises an electrolysis unit 250 arranged to receive a second CO₂ rich feed 201′, or the first CO₂ rich feed 201 of FIG. 2 comprising CO₂, or a portion thereof, and provide a CO-enriched feed stream 204, as well as second oxygen stream 206′. Suitably also, a portion of the second CO₂ rich feed 201′ may bypass the electrolysis unit 250 and combine with the CO-enriched feed stream 204 (not shown). A mixing unit or junction (not shown), serves to combine streams 202′ and 204 into second syngas feed 205, having a molar ratio of CO/CO₂ higher than 1, for instance 10 or higher, thus also enabling a more reactive syngas for methanol synthesis. A mixing unit or junction (not shown) combine a steam stream 207 with first oxygen stream 206 and/or second oxygen stream 206′, to provide an oxidant stream 208, which is fed to reforming system 100. The third ATR-based syngas 53 is suitably admixed to the second syngas feed 205 to provide the more reactive methanol feed gas as the inlet to methanol synthesis unit 220.

FIG. 4 shows the gasoline synthesis plant 200 as in FIG. 2, and further comprising an embodiment in which excess hydrogen stream 255 is produced internally. Hence, the methanol synthesis unit 220 is arranged to provide the excess hydrogen stream 255, and the reforming system 100 comprises a hydrogenation section 10, as shown in FIG. 1, which is further arranged to receive said excess hydrogen stream 255, i.e. said excess hydrogen stream 255 is supplied to said hydrogenation section 50. Excess hydrogen stream 255 may also be provided (not shown) to the upgrading section 240, for instance to a HDI reactor arranged therein.

### Example

TABLE 1
Comparison of product yield by LPG and
off-gas recycle in e-gasoline plant

| Paremeters | UoM | C1 | C2 |
|---|---|---|---|
| Main feed | — | First syngas combining H₂ | |
| | | from electrolysis, and CO₂ | |
| Electrolysis H₂ feed mass | % | 100 | 100 |
| flow | | | |
| CO₂ feed mass flow | % | 100 | 100 |
| ATR | — | No | Yes |
| Total syngas feed mass | % | 100 | 117.5 |
| flow to MeOH synthesis | | | |
| Methanol mass flow as | % | 100 | 117.5 |
| pure MeOH | | | |
| Gasoline mass flow | % | 100 | 117.5 |
| Hydrogen-efficiency in | % | 65 | 77 |
| methanol synthesis unit | | | |
| as H in CH₃OH per H in | | | |
| electrolysis H₂ feed to the | | | |
| plant battery limits (BL) | | | |
| C-efficiency of plant as C | % | 79 | 97 |
| in gasoline product per C | | | |
| in CO₂ feed to the plant | | | |
| BL | | | |
| Main equipment sizing | % | 100 | 100 |
| required in methanol loop | | | |
| and catalyst volume | | | |
| required in methanol | | | |
| synthesis unit | | | |

Results from a gasoline synthesis plant is shown in Table 1. The main syngas feed to the gasoline synthesis plant is a first syngas feed which combines a first CO₂ rich feed and first H₂ rich feed, the latter from electrolysis of water and/or steam. No other feed is used. C1 is the case, where LPG and off-gas by-products from the system are not utilized. In C2, all LPG and off-gas streams are recycled and reformed in an ATR to produce additional syngas as ATR-based syngas stream and then added to main syngas feed (first syngas feed) to the methanol synthesis unit (methanol synthesis loop). The methanol synthesis unit is arranged to provide an excess hydrogen stream which is fed to the hydrogenation section of the reforming system instead of providing the hydrogen from a hydrogen recovery section therein. As a result of the plant or process according to the present invention, intermediate methanol production is increased by roughly 20%. The final gasoline product is also increased by roughly 20% thus providing a significantly better yield of product from same amount of feed. Even though the methanol production rate increases by roughly 20%, the catalyst volume is the same as what is needed to convert solely the mixture of the first H₂ rich feed and the first CO₂ rich feed. The water formation in the methanol synthesis is essentially the same as for a base case where solely the mixture of the first H₂ rich feed and first CO₂ rich feed is being fed to the methanol synthesis loop. Hence, the boost in production by recycling via a reforming system of a by-product stream rich in paraffins, such as an LPG stream, and other off-gas streams, can be achieved without increasing size and duty considerably in a distillation section downstream the methanol synthesis reactor of the methanol synthesis loop. Furthermore, as shown in the antepenultimate line of the Table, the maximum stoichiometrically achievable hydrogen efficiency of the methanol synthesis loop increases from about 65% with solely said hydrogen and carbon dioxide feed to 75-80% due to reduced loss of hydrogen in water formation in the loop. Carbon efficiency (C-efficiency), as shown in the penultimate line of the table, is also significantly increased from about 80% to 97%. The above improvements are obtained without changing the main equipment size in the methanol synthesis unit, as shown in the last line of the table. The main equipment size of the methanol loop may also even be reduced.

In addition, the provision of excess hydrogen stream from the methanol synthesis loop provides high integration in the plant and process, since it can be used as hydrogen source for e.g. the HDI reactor of the upgrading section or for the hydrogenation section in the reforming system of the plant. At least a portion of the hydrogen required is thus sourced internally, rather than costly external sourcing from outside the battery limits of the plant. Furthermore, the provision of a methanol storage tank between the methanol synthesis unit and the gasoline synthesis section provides also a simple solution for coping with intermittent sources for producing the electricity required in e.g. upstream electrolysis. The methanol storage tank accumulates the methanol at low pressure, such as atmospheric pressure, thus enabling the use of inexpensive materials for such tank while also serving as buffer for any sudden variations in electricity due to the intermittent nature of the source producing it, such as wind and solar energy. The plant and process according to the present invention thus enables not only improving hydrogen (H) and carbon (C) efficiency of the plant, while improving performance and thereby reducing the size of the methanol synthesis unit, for instance a MeOH-loop, or at least without changing the main equipment size in the methanol synthesis unit, but at the same time provides a robust plant which copes with the sudden and often huge variations in electricity supply for e.g. electrolysis of water or steam into the hydrogen required in the syngas feed for methanol production, or oxygen produced in the electrolysis which is used as oxidant stream in the ATR.

The present invention has been described with reference to a number of embodiments and figures. However, the skilled person is able to select and combine various embodiments within the scope of the invention, which is defined by the appended claims. All documents referenced herein are incorporated by reference.

We claim:

1. A gasoline synthesis plant, comprising:
a first CO₂ rich feed supplying CO₂ to said plant, a first H₂ rich feed supplying H₂ to said plant, or a first syngas feed combining the first CO₂ rich feed and the first H₂ rich feed; or a second syngas feed supplying a carbon oxide and hydrogen to said plant;
a methanol synthesis unit, arranged to receive the first CO₂ rich feed and the first H₂ rich feed, or arranged to receive the first syngas feed, or arranged to receive the second syngas feed, and provide an effluent stream comprising methanol;
a gasoline synthesis section, arranged to receive at least a portion of the effluent stream comprising methanol, and provide a raw product containing hydrocarbons boiling in the gasoline range;
said gasoline synthesis section being arranged to provide a by-product stream rich in paraffins;
said gasoline synthesis plant further comprising a reforming system for reforming said by-product stream rich in paraffins, said reforming system comprising: a first reforming feed stream as said by-product stream rich in paraffins; an autothermal reformer (ATR) with no primary reformer arranged together with the ATR, the ATR being arranged to receive the by-product stream rich in paraffins and carry out an autothermal reforming step; and said reforming system providing an ATR-based syngas stream;
said gasoline synthesis plant further being arranged to feed at least a portion of said ATR-based syngas stream to the inlet of the methanol synthesis unit;
wherein said gasoline synthesis plant does not comprise a reforming unit arranged upstream of the methanol synthesis unit for providing said first or second syngas feed.

2. The gasoline plant according to claim 17, wherein:
the methanol synthesis unit is arranged to provide an excess hydrogen stream, and said upgrading section is arranged to receive: a portion of the first or second H₂ rich feed comprising H₂, and/or at least a portion of said excess hydrogen stream from the methanol synthesis unit;
and/or
the methanol synthesis unit is arranged to provide an excess hydrogen stream, and said reforming system is arranged to receive at least a portion of said excess hydrogen stream from the methanol synthesis unit.

3. The gasoline synthesis plant according to claim 18, wherein:
said HDI reactor and/or HCR reactor of said upgrading section is arranged to receive: a portion of the first or second H₂ rich feed comprising H₂, and/or at least a portion of said excess hydrogen stream from the methanol synthesis unit.

4. The gasoline synthesis plant according to claim 1, wherein said reforming system is further arranged for said first reforming feed being less than 15 wt % of said raw product containing hydrocarbons boiling in the gasoline range, or less than 15 wt % of said gasoline product stream.

5. The gasoline synthesis plant according to claim 1, further comprising:
an electrolysis section comprising:
an electrolysis unit arranged to receive a water feedstock, and provide: a second H₂ rich feed comprising H₂; and a first oxygen stream;
and/or
an electrolysis unit arranged to receive a second CO₂ rich feed, or said first CO₂ rich feed comprising CO₂, or a portion thereof, and provide a CO-enriched feed and a second oxygen stream; and
a mixing unit or junction to combine the first or second H₂ rich feed comprising H₂, with the CO-enriched feed, and provide said second syngas feed.

6. The gasoline synthesis plant according to claim 5, wherein the plant is configured to
combine a steam stream with the first oxygen stream to provide an oxidant stream; and wherein the ATR of the reforming system is arranged to receive said oxidant stream; and/or
combine the first and/or second (206′) oxygen streams, with said steam stream to provide an oxidant stream.

7. The gasoline synthesis plant according to claim 1, further comprising:
a thermal decomposition unit, arranged to receive a biomass feedstock to provide a crude syngas feed, and a crude syngas purification section arranged to receive the crude syngas feed and to provide said second syngas feed;
or
a reverse water gas shift unit, arranged to receive a portion of said first CO₂ rich feed comprising CO₂ and a portion of said first H₂ rich feed comprising H₂, to provide a rWGS syngas feed;
wherein the plant is configured to combine the rWGS syngas feed with the remaining portion of: said first CO₂ rich feed comprising CO₂ and said first H₂ rich feed comprising H₂, and provide said second syngas feed.

8. The gasoline synthesis plant according to claim 1, wherein a methanol storage tank is arranged between said methanol synthesis unit and said gasoline synthesis section, for storing at least a portion of the effluent stream comprising methanol.

9. The gasoline synthesis plant according to claim 1, wherein the at least a portion of said ATR-based syngas stream, is arranged to be fed to the inlet of the methanol synthesis unit in admixture with said CO₂ rich feed and/or said H₂ rich feed, or in admixture with said first syngas feed, or in admixture with said second syngas feed.

10. The gasoline synthesis plant according to claim 1, wherein the methanol synthesis unit is arranged for the ATR-based syngas stream being up to up to 50% by volume basis.

11. The gasoline synthesis plant according to claim 1, wherein the methanol synthesis unit is a methanol synthesis loop, which comprises:
optionally, a cleaning section, arranged to receive said CO₂ rich feed and/or said H₂ rich feed, or said first syngas feed; or said second syngas feed, thereby providing a cleaned methanol syngas feed;
a methanol reactor arranged to receive the cleaned methanol syngas feed and to produce a raw methanol effluent stream;
a first separator arranged to receive the raw methanol effluent stream, and to produce a bottom stream as said effluent stream comprising methanol, and said first separator also being arranged to produce an overhead recycle stream;
a recycle compressor arranged to recycle the overhead recycle stream to the methanol reactor.

12. The gasoline synthesis plant of claim 11, wherein the methanol synthesis unit is configured
to combine the ATR-based syngas stream with the overhead recycle stream prior to being fed to the inlet of the methanol synthesis unit in admixture with said CO₂ rich feed and/or said H₂ rich feed, or in admixture with said first syngas feed, or in admixture with said second syngas feed.

13. The gasoline synthesis plant according to claim 11, wherein the methanol synthesis unit further comprises: a conduit for withdrawing a portion of said overhead recycle stream as a fuel gas, and providing therefrom said excess hydrogen stream from the methanol synthesis unit wherein said excess hydrogen stream is provided by the methanol synthesis unit further comprising a hydrogen recovery unit arranged to receive said fuel gas and provide a hydrogen-rich stream as said excess hydrogen stream, the hydrogen recovery unit being at least one of: a gas separator such as a gas scrubber, a membrane unit, and a pressure swing adsorption unit.

14. The gasoline synthesis plant according to claim 1, wherein the by-product stream rich in paraffins is a stream rich in propane and/or butane,
and/or
wherein the by-product stream rich in paraffins is a naphtha stream;
and/or
wherein the plant is arranged to provide one or more off-gas streams, said one or more off-gas streams being a waste-gas stream rich in CO₂, H₂, CH₄, and wherein said reforming system is arranged to receive at least a portion of said one or more off-gas stream(s).

15. The gasoline synthesis plant according to claim 1, wherein the plant further comprises in said reforming system a separation section, arranged to receive at least a portion of said ATR-based syngas stream as a first ATR-based syngas stream and separate it into at least a second ATR-based syngas stream and a process condensate;
wherein the plant further comprises in said reforming system a hydrogen recovery section, said hydrogen recovery section being arranged to receive at least a portion of the second ATR-based syngas stream and provide at least a hydrogen-rich stream and a fourth ATR-based syngas stream; and
wherein at least a portion of the second ATR-based syngas stream and at least a portion of the fourth ATR-based syngas stream are arranged to be combined into third ATR-based syngas stream.

16. A process for gasoline synthesis of a first CO₂ rich feed comprising CO₂, and a first H₂ rich feed comprising H₂, or from a first syngas feed which combines said first CO₂ rich feed and said first H₂ rich feed; or from a second syngas feed comprising a carbon oxide and hydrogen, said process comprising the steps of:
providing a gasoline synthesis plant according to claim 1;
supplying CO₂ rich feed and H₂ rich feed, or said first syngas feed, or said second syngas feed, to the methanol synthesis unit, and providing an effluent stream comprising methanol;
supplying at least a portion of the effluent stream comprising methanol from the methanol synthesis unit to the gasoline synthesis section, and providing a raw product containing hydrocarbons boiling in the gasoline range;
supplying at least a portion of the raw product from the gasoline synthesis section to optional an upgrading section, and providing a gasoline product stream; said upgrading section comprising: a hydroisomerisation (HDI) reactor, thereby providing said gasoline product stream;
withdrawing from the gasoline section and/or the optional upgrading section a first stream rich in paraffins; and/or second stream from the optional upgrading section;
supplying at least a portion of a first stream as said first stream rich in paraffins, to reforming system, performing autothermal reforming step in the ATR, and providing an ATR-based syngas stream;
supplying at least a portion of said ATR-based syngas stream to the inlet of the methanol synthesis unit, in admixture with said CO₂ rich feed and/or said H₂ rich feed, or in admixture with said first syngas feed; or in admixture with said second syngas feed;
wherein the process does not comprise a primary reforming step in connection with said autothermal reforming step;
wherein the process does not comprise steam reforming of a hydrocarbon feed gas for providing said first syngas feed or said second syngas feed.

17. The plant of claim 1 comprising an upgrading section arranged to receive at least a portion of the raw product from the gasoline synthesis section, and provide a gasoline product stream; said upgrading section comprising: a hydroisomerisation reactor, thereby providing said gasoline product stream.

18. The plant of claim 17, wherein said upgrading section comprises a hydrocracking reactor.

19. The plant of claim 1, wherein said reforming system comprises a hydrogenation section, and wherein said hydrogenation section is arranged to receive said excess hydrogen stream.

20. The plant of claim 3, wherein the gasoline synthesis section comprises: a methanol-to-gasoline section, and a downstream distillation section comprising a de-ethanizer and a LPG-splitter.

21. The plant of claim 5, wherein the electrolysis unit is arranged to provide: a second H₂ rich feed comprising H₂; optionally as said first H₂ rich feed comprising H₂, or a portion thereof as said first H₂ rich feed; and a first oxygen stream);

22. The plant of claim 11, wherein the cleaning section comprises a desulphurisation section, and wherein the cleaned methanol syngas feed is a desulfurized methanol syngas feed, and wherein the methanol synthesis loop comprises a second separator from which an off-gas is generated.
